(19) United States
(12) Patent Application Publication
(10) Pub. No.: US 2026/0279504 A1
(43) Pub. Date: Sep. 17, 2026

(54) INFORMATION PROVISION METHOD, INFORMATION PROVISION SYSTEM, AND RECORDING MEDIUM

(71) Applicant: Panasonic Intellectual Property Management Co., Ltd., Osaka (JP)

(72) Inventors: KIYOTAKA TSUJI, Osaka (JP); KENJI KONDO, Kyoto (JP); KAORI SUZUKI, Osaka (JP)

(21) Appl. No.: 19/674,182

(22) Filed: May 12, 2026

Related U.S. Application Data
(63) Continuation of application No. PCT/JP2024/042715, filed on Dec. 3, 2024.

(30) Foreign Application Priority Data
Dec. 11, 2023 (JP) 2023-208336

(51) Int. Cl. G16C 20/10

(52) U.S. Cl. CPC G16C 20/10

(57) ABSTRACT

An information provision method, the information provision method being executed by a computer, the method including: acquiring standard ingredient information indicating a standard ingredient and a quantity of the standard ingredient, deriving, from reference ingredients, substitute ingredient information indicating at least one substitute ingredient serving as a replacement for the standard ingredient and a quantity of the at least one substitute ingredient, based on the quantity of the standard ingredient, a content of a chemical component contained in the standard ingredient, and a content of a chemical component contained in each of the reference ingredients, and outputting the substitute ingredient information.

### BACKGROUND

### 1. TECHNICAL FIELD

The present disclosure relates to an information provision method, an information provision system, and a recording medium that provide information regarding food materials.

### 2. DESCRIPTION OF THE RELATED ART

Japanese Patent No. 7095267 discloses an information processing apparatus that allows a user to grasp the reproducibility score of a recipe by taking into account individual differences in the ingredients included in the recipe or to grasp the reproducibility score of a recipe in a case where cooking is performed using another ingredient substituted for an ingredient included in the recipe.

Japanese Unexamined Patent Application Publication No. 2022-102185 discloses a recipe provision apparatus that effectively supports cooking for a user. This recipe provision apparatus outputs change recipe information, which is substitute information regarding recipe information, in a case where change request information for changing the recipe information is accepted.

### SUMMARY

However, based on the technologies described in Japanese Patent No. 7095267 and Japanese Unexamined Patent Application Publication No. 2022-102185, there may be a case where a food cannot be prepared in a case where one or more of the ingredients that are needed to prepare the food are unavailable.

One non-limiting and exemplary embodiment provides an information provision method and the like that enable a food or a food equivalent thereto to be prepared without using one or more of the ingredients needed to prepare the food.

In one general aspect, the techniques disclosed here feature an information provision method, the information provision method being executed by a computer, the method including: acquiring standard ingredient information indicating a standard ingredient and a quantity of the standard ingredient, deriving, from reference ingredients, substitute ingredient information indicating at least one substitute ingredient serving as a replacement for the standard ingredient and a quantity of the at least one substitute ingredient, based on the quantity of the standard ingredient, a content of a chemical component contained in the standard ingredient, and a content of a chemical component contained in each of the reference ingredients, and outputting the substitute ingredient information.

An information provision method and the like according to the present disclosure make it possible to provide information that enables a food or a food equivalent thereto to be prepared without using one or more of the ingredients needed to prepare the food.

It should be noted that general or specific embodiments may be implemented as a system, a method, an integrated circuit, a computer program, a storage medium, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a diagram illustrating an example in which a food is prepared using ingredients;

FIG. 2 is a diagram illustrating an example in which the food is prepared using ingredients that are partially different from those illustrated in FIG. 1;

FIG. 3 is a schematic diagram illustrating an information provision system according to an embodiment;

FIG. 4 is a block diagram illustrating a functional configuration of the information provision system according to the embodiment;

FIG. 5 is a diagram illustrating an example of recipe information corresponding to foods stored in a recipe database;

FIG. 6 is a diagram illustrating an example of reference ingredient information stored in an ingredient database;

FIG. 7 is a diagram illustrating an example of standard recipe information regarding a standard recipe for a food subject to a recipe change;

FIG. 8 is a diagram illustrating an example of candidate information displaying candidates for change for the standard recipe;

FIG. 9 is a diagram illustrating an example of a screen on which change request information is displayed by an output unit of a terminal device, the change request information being information reflecting a user's request for the candidate information in FIG. 8;

FIG. 10 is a diagram illustrating an example of a chemical component list generated for ingredients on the basis of the change request information;

FIG. 11 is a diagram illustrating examples of substitute ingredient candidates serving as possible replacements for standard ingredients;

FIG. 12 is a diagram illustrating an example of substitute recipe information including substitute ingredient information;

FIG. 13 is a diagram illustrating the reproducibility scores of the contents of chemical components in substitute ingredients relative to the standard ingredients;

FIG. 14 includes diagrams illustrating examples of screens displaying substitute ingredients for standard ingredients and the reproducibility scores of the contents of the chemical components;

FIG. 15 is a diagram illustrating an example of ingredient information that is not stored in a database;

FIG. 16 is a diagram illustrating an example of other substitute ingredient candidates;

FIG. 17 is a diagram illustrating an example of substitute recipe information using other substitute ingredients;

FIG. 18 is a diagram illustrating information regarding other substitute ingredients;

FIG. 19 is a diagram illustrating an example of a screen displayed in a case where no substitute ingredient is available;

FIG. 20 is a diagram illustrating an operation flowchart of an information provision system according to the embodiment;

FIG. 21 is a block diagram illustrating a functional configuration of an information provision system according to a modification of the embodiment;

FIG. 22 is a diagram illustrating an operation flowchart of the information provision system according to the modification of the embodiment;

FIG. 23 is a diagram illustrating an example of a substitute ingredient list including substitute ingredient candidates; and

FIG. 24 is a diagram illustrating an example of a screen for selecting substitute ingredients from the substitute ingredient list.

### DETAILED DESCRIPTIONS

### Underlying Knowledge Forming Basis of the Present Disclosure

Some foods are prepared using ingredients.

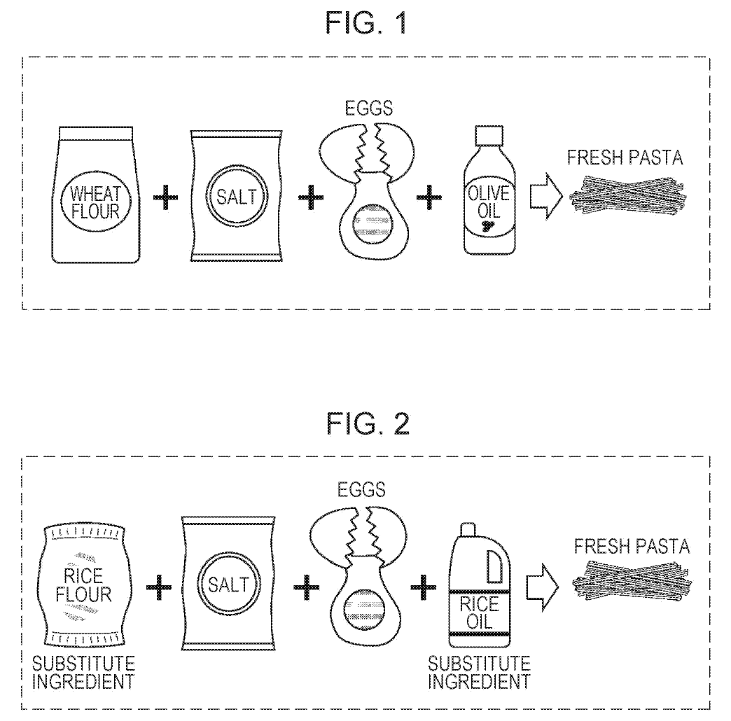

FIG. 1 is a diagram illustrating an example in which a food is prepared using ingredients.

FIG. 1 illustrates an example of preparing a food called fresh pasta using ingredients that include wheat flour, salt, eggs, and olive oil. In the example illustrated in FIG. 1, all ingredients for the food need to be present in order to prepare the food.

FIG. 2 is a diagram illustrating an example in which the food is prepared using ingredients that are partially different from those illustrated in FIG. 1.

FIG. 2 illustrates an example in which the food called fresh pasta is prepared using substitute ingredients that are different from those illustrated in FIG. 1, such as rice flour, salt, eggs, and rice oil. As illustrated in FIG. 2, even in a case where, for example, some of the ingredients needed to prepare the food are unavailable, the food or a food equivalent thereto can be prepared by using substitute ingredients. Even in a case where there is an ingredient that cannot be used because of an allergy (a wheat allergy in the example illustrated in FIG. 2), the food or a food equivalent thereto can be prepared in an allergen-free manner. In addition, even in a case where some ingredients are intentionally excluded because they are not desired due to personal preferences or a case where other ingredients are intentionally included because the use of regional specialties is desired, the food or a food equivalent thereto can be prepared.

Note that the example in which the food is a processed ingredient that is not yet cooked is mentioned above; however, the food is not limited thereto and may be a cooked food product (in other words, a dish). For example, ingredients may be fresh pasta, eggs, powdered cheese, and bacon, and a food prepared using the ingredients may be carbonara. That is, a food may be any food prepared using ingredients, including foods such as processed ingredients that are not yet cooked and foods that are cooked food products.

However, the present inventors have identified three main issues with the use of substitute ingredients.

First, the sensory evaluations for foods prepared using substitute ingredients will be lower than those for foods prepared without using substitute ingredients, and the use of substitute ingredients in a food may result in a decline in the food's sensory quality.

Second, foods prepared using substitute ingredients may be different in terms of one or more nutrients that foods prepared without using substitute ingredients have.

Third, it is not always sufficient to use the same amount of a substitute ingredient as an ingredient being substituted, and the amount of the substitute ingredient needs to be adjusted in accordance with the ingredient being substituted or the food prepared using the substitute ingredient.

Regarding the above-mentioned three issues, the present inventors have completed an information provision method and the like according to the present disclosure that make it possible to prepare a food or a food equivalent thereto without using some ingredients of the ingredients needed to prepare the food, by focusing on the chemical components contained in the ingredients. In the following, examples of an information provision method and the like according to an aspect of the present disclosure will be illustrated.

An information provision method according to Example 1 is an information provision method executed by a computer, the method including: acquiring standard ingredient information indicating a standard ingredient and a quantity of the standard ingredient, deriving, from reference ingredients, substitute ingredient information indicating at least one substitute ingredient serving as a replacement for the standard ingredient and a quantity of the at least one substitute ingredient, based on the quantity of the standard ingredient, a content of a chemical component contained in the standard ingredient, and a content of a chemical component contained in each of the reference ingredients, and outputting the substitute ingredient information.

In this manner, even if one or more of the ingredients needed to prepare a food are not used, information can be provided that makes it possible to prepare the food or a food equivalent thereto by deriving a substitute ingredient and the quantity of the substitute ingredient based on the quantity of a standard ingredient, the content of a chemical component contained in the standard ingredient, and the content of a chemical component contained in each of the reference ingredients.

An information provision method according to Example 2 is the information provision method according to Example 1 in which in the deriving the substitute ingredient information, a reference ingredient that contains the chemical component contained in the standard ingredient may be selected, as the at least one substitute ingredient, from among the reference ingredients, and the quantity of the at least one substitute ingredient may be derived based on the quantity of the standard ingredient, the content of the chemical component contained in the standard ingredient, and the content of the chemical component contained in the at least one substitute ingredient.

In this manner, a substitute ingredient can be appropriately selected by selecting, as a substitute ingredient, a reference ingredient that contains a chemical component contained in a standard ingredient. This makes it possible to provide information that enables a food or a food equivalent thereto to be prepared without using one or more of the ingredients needed to prepare the food.

An information provision method according to Example 3 is the information provision method according to Example 1 or 2 in which the chemical component contained in the standard ingredient may include one or more predetermined chemical components contained in the standard ingredient, and in the deriving the substitute ingredient information, the at least one substitute ingredient may be derived from the reference ingredients so as to contain all of the one or more predetermined chemical components contained in the standard ingredient, and the quantity of the at least one substitute ingredient may be derived based on the quantity of the standard ingredient, contents of all of the one or more predetermined chemical components contained in the standard ingredient, and contents of all of the one or more predetermined chemical components contained in the at least one substitute ingredient.

In this manner, a substitute ingredient can be appropriately derived by deriving a substitute ingredient that contains all of the one or more predetermined chemical components contained in a standard ingredient. This makes it possible to provide information that enables a food or a food equivalent thereto to be prepared without using one or more of the ingredients needed to prepare the food.

An information provision method according to Example 4 is the information provision method according to Example 3 in which the at least one substitute ingredient may contain a different chemical component from the one or more predetermined chemical components contained in the standard ingredient.

This makes it possible to provide an ingredient that contains a different chemical component from that of a standard ingredient as information regarding a substitute ingredient. This makes it possible to provide information that enables a food or a food equivalent thereto to be prepared without using one or more of the ingredients needed to prepare the food.

An information provision method according to Example 5 is the information provision method according to any one of Examples 1 to 4 in which the standard ingredient information may be included in standard recipe information indicating a standard recipe for a food prepared using the standard ingredient, information indicating the content of the chemical component contained in each of the reference ingredients may be included in reference ingredient information, the substitute ingredient information may be included in substitute recipe information indicating a substitute recipe for the food prepared using the at least one substitute ingredient, and in the deriving the substitute ingredient information, the substitute recipe information may be derived based on the standard recipe information and the reference ingredient information.

In this manner, by deriving substitute recipe information based on standard recipe information and reference ingredient information, a substitute recipe that corresponds to a standard recipe and in which one or more standard ingredients in the standard recipe are not used can be provided.

An information provision method according to Example 6 is the information provision method according to Example 5 in which the standard ingredient information may further include information indicating a sensory evaluation associated with the standard ingredient, the reference ingredient information may further include information indicating a sensory evaluation associated with each of the reference ingredients, the substitute ingredient information may further include information indicating a sensory evaluation associated with the substitute ingredient, and in the deriving the substitute ingredient information, a reference ingredient that includes the sensory evaluation associated with the standard ingredient may be derived from the reference ingredients as the at least one substitute ingredient to derive the substitute ingredient information.

This makes it possible to derive, as a substitute ingredient, a reference ingredient having a sensory evaluation associated with a standard ingredient and to provide information regarding the substitute ingredient having this sensory evaluation. This makes it possible to provide information that enables a food or a food equivalent thereto to be prepared without using one or more of the ingredients needed to prepare the food.

An information provision method according to Example 7 is the information provision method according to Example 6 in which the sensory evaluation associated with the standard ingredient may include one or more predetermined sensory evaluations associated with the standard ingredient, and in the deriving the substitute ingredient information, the at least one substitute ingredient may be derived from the reference ingredients so as to include all of the one or more predetermined sensory evaluations associated with the standard ingredient.

This makes it possible to derive, as a substitute ingredient, a reference ingredient having one or more predetermined sensory evaluations associated with a standard ingredient and to provide information regarding the substitute ingredient having this sensory evaluation. This makes it possible to provide information that enables a food or a food equivalent thereto to be prepared without using one or more of the ingredients needed to prepare the food.

An information provision method according to Example 8 is the information provision method according to any one of Examples 1 to 7 in which in the deriving the substitute ingredient information, a reproducibility score indicating a degree of reproducibility of the standard ingredient may be derived, and the at least one substitute ingredient is derived based on the reproducibility score.

By deriving a substitute ingredient based on the above-mentioned reproducibility score, information regarding a substitute ingredient having a high reproducibility score relative to the standard ingredient can be provided.

An information provision method according to Example 9 is the information provision method according to Example 8 in which the reproducibility score may be a ratio of a value obtained based on the quantity of the substitute ingredient and the content of the chemical component contained in the substitute ingredient to a value obtained based on the quantity of the standard ingredient and the content of the chemical component contained in the standard ingredient.

By deriving a substitute ingredient based on the above-mentioned reproducibility score, information regarding a substitute ingredient having a high reproducibility score relative to the standard ingredient can be provided.

An information provision method according to Example 10 is the information provision method according to Example 8 in which the substitute ingredient may include substitute ingredients, and the reproducibility score may be a ratio of a sum of values obtained for each of the substitute ingredients, each value being obtained based on the quantity of the substitute ingredient and the content of the chemical component contained in the substitute ingredient, to a value obtained based on the quantity of the standard ingredient and the content of the chemical component contained in the standard ingredient.

By deriving a substitute ingredient based on the above-mentioned reproducibility score, information regarding a substitute ingredient having a high total reproducibility score relative to the standard ingredient can be provided.

An information provision method according to Example 11 is the information provision method according to any one of Examples 1 to 10 in which in the deriving the substitute ingredient information, substitute ingredient candidates serving as possible replacements for the standard ingredient may be extracted from the reference ingredients, selection of at least one substitute ingredient serving as a substitute for the standard ingredient from among the substitute ingredient candidates may be accepted, and the substitute ingredient information indicating the substitute ingredient and the quantity of the substitute ingredient may be derived based on the at least one substitute ingredient that is selected and accepted.

This allows, for example, the user to prepare a food using their desired substitute ingredient.

An information provision system according to Example 12 includes a server that derives a substitute ingredient serving as a replacement for a standard ingredient, and the server acquires information indicating the standard ingredient and a quantity of the standard ingredient and derives at least one substitute ingredient from reference ingredients and information indicating a quantity of the at least one substitute ingredient, based on the quantity of the standard ingredient, a content of a chemical component contained in the standard ingredient, and a content of a chemical component contained in each of the reference ingredients.

In this manner, even if one or more of the ingredients needed to prepare a food are not used, information can be provided that makes it possible to prepare the food or a food equivalent thereto by deriving a substitute ingredient and the quantity of the substitute ingredient based on the quantity of a standard ingredient, the content of a chemical component contained in the standard ingredient, and the content of a chemical component contained in each of reference ingredients.

A program according to Example 13 is a program that causes a computer to execute the information provision method according to any one of Examples 1 to 12.

This program makes it possible to execute the above-mentioned information provision method.

In the following, an information provision method, an information provision system, and the like according to an aspect of the present disclosure will be specifically described with reference to the drawings.

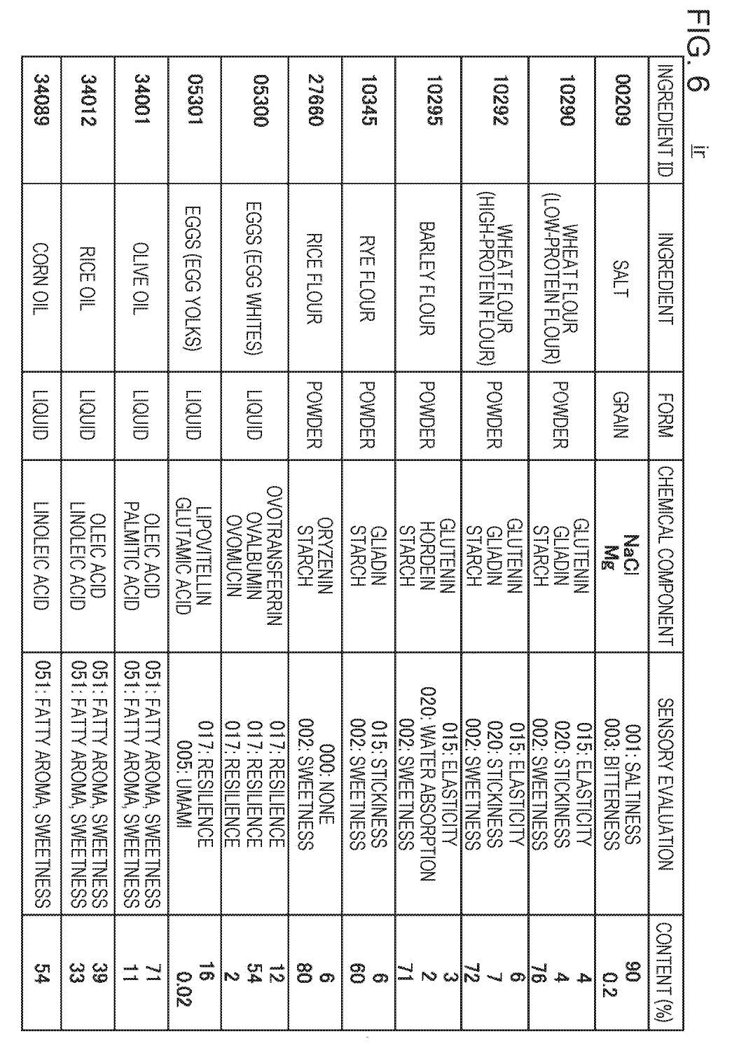

Note that each of the embodiments described below is a specific example of the present disclosure. For example, numerical values, shapes, materials, constituent elements, arrangement positions and connection forms of the constituent elements, steps, sequences of steps indicated in the following embodiments are examples and are not intended to limit the present disclosure. In addition, among the constituent elements in the following embodiments, constituent elements that are not described in the independent claims that indicate the highest level concept are described as optional constituent elements. "fatty aroma, sweetness" in FIGS. 6 to 11, FIGS. 13 to 16, and FIGS. 18 to 19 may also mean "fatty aroma and sweetness".

### Embodiments

### Overall Configuration of Information Provision System

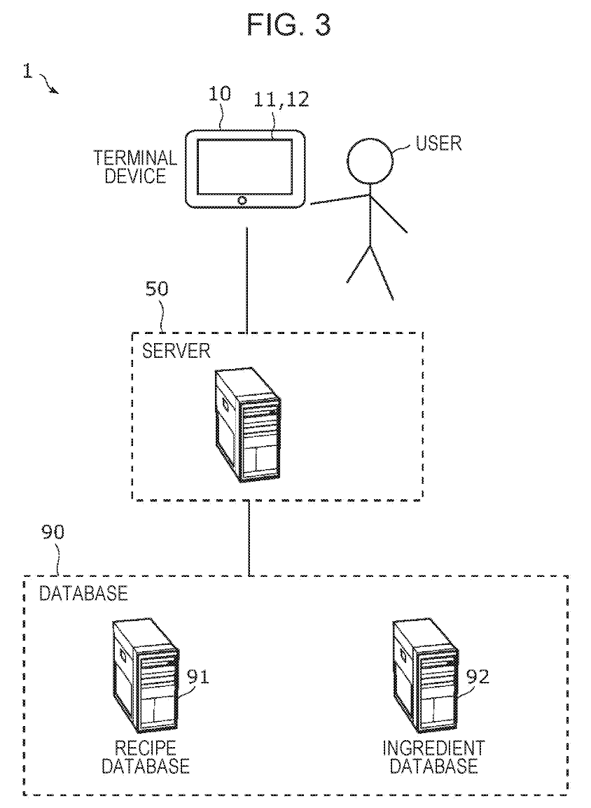

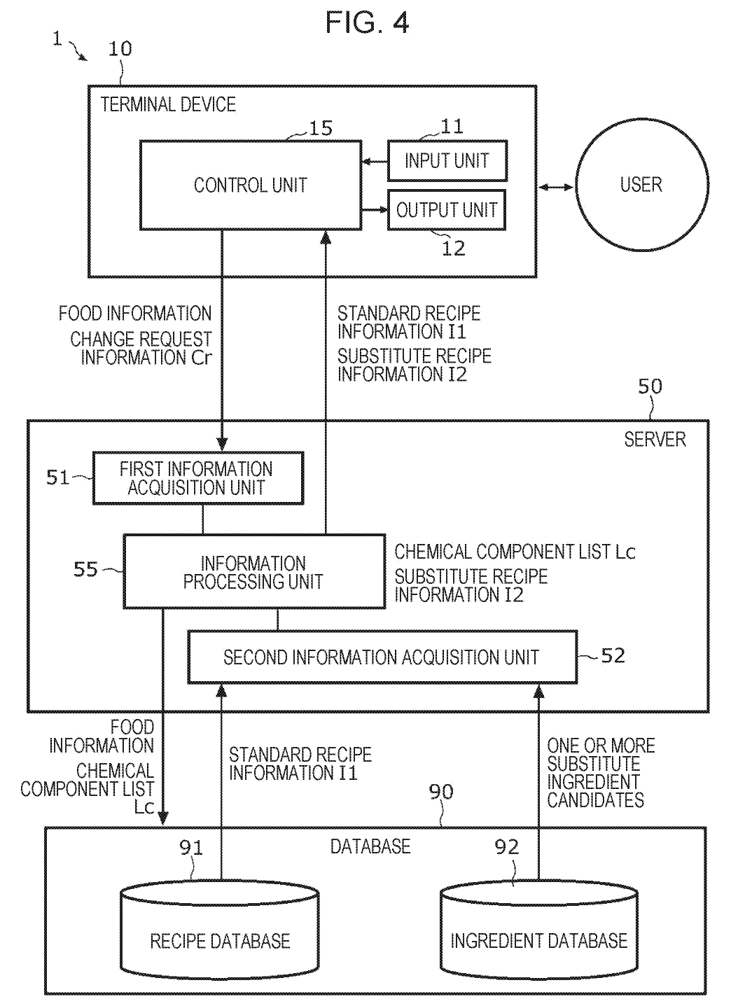

The overall configuration of an information provision system according to an embodiment will be described with reference to FIGS. 3 and 4.

FIG. 3 is a schematic diagram illustrating an information provision system 1 according to the embodiment. FIG. 4 is a block diagram illustrating a functional configuration of the information provision system 1 according to the embodiment.

The information provision system 1 includes a terminal device 10, a server 50, and a database 90. FIGS. 3 and 4 also illustrate users of the information provision system 1. The terminal device 10 accepts various types of inputs from the user and provides various type of information to the user.

The terminal device 10 and the server 50 are connected for communication via a communication network. The server 50 and the database 90 are connected for communication via a communication network. Note that the database 90 may be provided within the server 50 instead of outside the server 50.

The terminal device 10 exchanges various types of information with the server 50 and the user of the information provision system 1. Examples of the user include a cook who performs cooking, a culinary researcher who develops recipes, and a researcher who develops cooking apparatuses. The cook may be a person who cooks in a restaurant or a person who cooks in a culinary school or at home.

Examples of the terminal device 10 include a tablet computer, a desktop computer, a notebook computer, and a smartphone. The terminal device 10 includes an input unit 11, an output unit 12, and a control unit 15.

The control unit 15 has a function of controlling the input unit 11 and the output unit 12. The control unit 15 also has a communication function for communicating with the server 50.

The input unit 11 is a device having a function for the user to input necessary information into the terminal device 10. The input unit 11 may be one of a touch panel, a mouse, a keyboard, or a microphone (voice input) or any combination thereof. The terminal device 10 accepts various types of information from the user via the input unit 11.

The output unit 12 is a device having a function of presenting necessary information to the user and also having a function of presenting, to the user, the need to make a decision and input the result of the decision. The output unit 12 may be one of a display device, a printer, or a speaker or any combination thereof. Examples of the display device include a liquid crystal display and a CRT monitor. The control unit 15 causes the output unit 12 to output various types of information transmitted from the server 50. That is, the terminal device 10 uses the output unit 12 to provide various types of information to the user. The input unit 11 and the output unit 12 may be configured as a touch panel. The terminal device 10 does not necessarily include the input unit 11, and the terminal device 10 and the input unit 11 may be connected by wire or wirelessly. The terminal device 10 does not necessarily include the output unit 12, and the terminal device 10 and the output unit 12 may be connected by wire or wirelessly.

At the stage when the user begins to use the information provision system 1, the user inputs food information into the terminal device 10. The food information is information regarding the food desired by the user. In other words, the food information is information regarding the food to be prepared by the user. In other words, the food information is information regarding the food subject to a recipe change. The terminal device 10 transmits the received food information to the server 50. The terminal device 10 also receives, for example, standard recipe information I1 and substitute recipe information I2 from the server 50. The standard recipe information I1 and the substitute recipe information I2 will be described below.

The server 50 is a device that exchanges information with the terminal device 10 and the database 90. The server 50 according to the present embodiment derives substitute ingredients serving as replacements for ingredients used to prepare a food. The server 50 may include, for example, a computer and may be installed in a building of a management company that operates the server 50.

As illustrated in FIG. 4, the server 50 includes a first information acquisition unit 51, a second information acquisition unit 52, and an information processing unit 55. The information processing unit 55 has a function of controlling the first information acquisition unit 51 and the second information acquisition unit 52. The information processing unit 55 also has a communication function for communicating with the terminal device 10 and the database 90. The server 50 stores a program for the information processing unit 55 to perform information processing (data processing). Individual constituent elements included in the server 50 may be software functions fulfilled by executing programs stored in the server 50.

The first information acquisition unit 51 acquires information transmitted from the terminal device 10. The second information acquisition unit 52 acquires information transmitted from the database 90. For example, the first information acquisition unit 51 acquires food information, recipe change request information, and the like transmitted from the terminal device 10. The second information acquisition unit 52 acquires standard recipe information I1, information regarding substitute ingredient candidates, and the like transmitted from the database 90. Note that a single information acquisition unit may include the first information acquisition unit 51 and the second information acquisition unit 52.

The database 90 is, for example, a computer such as a database server. The database 90 includes a recipe database 91 and an ingredient database 92. The recipe database 91 stores data regarding pieces of recipe information. The ingredient database 92 stores data regarding pieces of reference ingredient information ir. The database 90 retrieves information from the information stored in the recipe database 91 and the ingredient database 92 in response to a request from the server 50 and transmits the information to the server 50.

Note that the database 90 may be constituted by a computer different from the server 50 or may be constituted by one or more computers using virtualization technology. The database 90 may be constituted by the same computer as the server 50. The database 90 may be big data that can be used for purposes.

### Detailed Configuration of Information Provision System

The detailed configuration of the information provision system 1 will be described with reference to FIGS. 4 to 19.

The terminal device 10 illustrated in FIG. 4 accepts food information from the user. In the present embodiment, an example will be described in which a food subject to a recipe change is "fresh pasta". In this example, the food information may indicate that the food is "fresh pasta". The terminal device 10 transmits the food information received from the user to the server 50.

The server 50 acquires the food information transmitted from the terminal device 10 and transmits the acquired food information to the database 90.

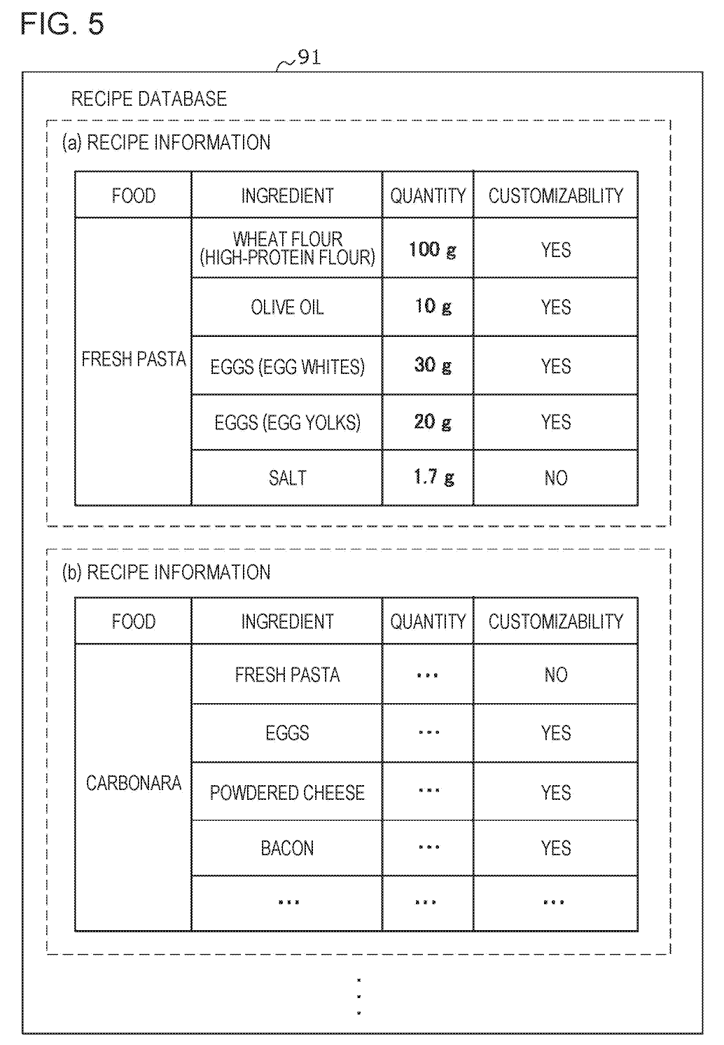

FIG. 5 is a diagram illustrating an example of recipe information corresponding to foods stored in the recipe database 91.

FIG. 5 illustrates pieces of recipe information stored in the recipe database 91. Among the pieces of recipe information, recipe information labeled (a) is recipe information corresponding to "fresh pasta", and recipe information labeled (b) is recipe information corresponding to "carbonara". Recipe information includes information regarding a food, ingredients needed to prepare the food, and the quantity of each ingredient. The recipe information according to the present embodiment includes, for each of the ingredients, information indicating whether or not the ingredient may be changed to other ingredients. The information indicating whether or not the ingredient may be changed to other ingredients may also be called ingredient customizability information.

Ingredients are materials needed to prepare foods. An ingredient may be a material itself, a processed ingredient obtained by processing materials to some extent, or a seasoning. A food is prepared using ingredients. As mentioned above, a food may be a processed ingredient that is not yet cooked or a cooked food product (in other words, a dish).

FIG. 6 is a diagram illustrating an example of the reference ingredient information ir stored in the ingredient database 92.

The reference ingredient information ir is ingredient information used as a reference to generate the standard recipe information I1 and the substitute recipe information I2. The reference ingredient information ir includes information regarding identification numbers (IDs) of ingredients , information regarding the ingredients (names of the ingredients), information regarding the forms of the ingredients, information regarding chemical components (names of the chemical components) contained in the ingredients, information regarding the contents of the chemical components contained in the ingredients, and information regarding the sensory evaluation of the ingredient. Note that the reference ingredient information ir does not necessarily need to be stored in a database. Information regarding relevant ingredients can be acquired in advance, and information regarding the chemical components and quantities can be extracted from the information regarding the ingredients.

The information regarding the forms of the ingredients is information indicating the physical state of each ingredient such as powder, liquid, or solid.

The information regarding the chemical components in the ingredient corresponds to one or more predetermined chemical components contained in the ingredient. For example, the one or more predetermined chemical components are important chemical components that characterize the ingredient or chemical components having high content levels, among the chemical components contained in the ingredient. There may be one or more predetermined chemical components listed in the reference ingredient information ir.

Information regarding sensory evaluation is information indicating the sensory evaluations corresponding to the chemical components. The sensory evaluations may be made based on any of taste, smell, or touch. Taste may be a sensation related to any of sweetness, bitterness, sourness, umami, or saltiness. Smell may be any of olfaction for a sweet aroma, olfaction for a bitter-associated aroma, olfaction for a fatty aroma, or olfaction for a sour aroma. Touch may also be called texture. Texture may be any of the following: texture related to viscosity, texture related to elasticity (firmness), texture related to resilience, texture related to stickiness, or texture related to water absorption. Human sensory perception has a close relationship with the chemical components contained in ingredients.

FIG. 6 illustrates that an ingredient "wheat flour (high-protein flour in this example)" having an ingredient ID (identification number) of "10292" has an ingredient form of powder and contains predetermined chemical components "glutenin, gliadin, starch". FIG. 6 also illustrates that the contents of "glutenin, gliadin, starch" are 6%, 7%, and 72%, respectively. Note that the content of each chemical component is the content of the chemical component when the total content of all the chemical components contained in the ingredient is 100, and the content is expressed as a percentage (%) in FIG. 6. FIG. 6 also illustrates that the ingredient "wheat flour" is strongly associated with "texture related to elasticity, texture related to stickiness, sensory perception related to sweetness".

Based on the food information transmitted from the server 50, the database 90 extracts information related to a recipe for the food, which is subject to a recipe change, from the recipe database 91 and the ingredient database 92 to generate the standard recipe information I1.

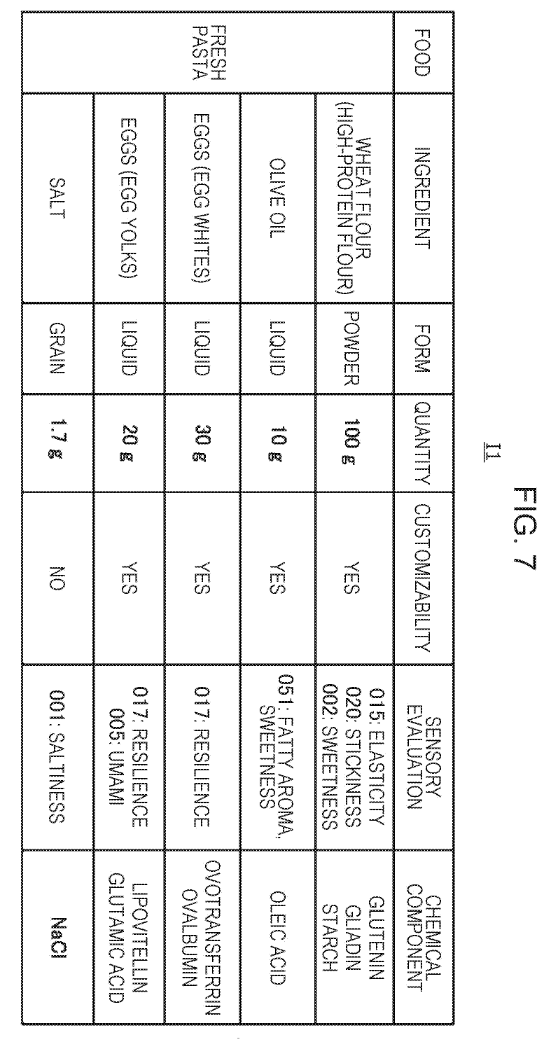

FIG. 7 is a diagram illustrating an example of the standard recipe information I1. FIG. 7 illustrates that the ingredients included in the standard recipe information I1 are used to prepare the food indicated by that standard recipe information I1. Each of the ingredients used may be an ingredient that is initially set as a commonly used ingredient or may be any ingredient that is set by the user. Each ingredient may also be determined based on a commercially available cookbook, information from a chef, or both. Some or all of the information included in the standard recipe information I1 may be interpreted as information that is taken into account when the user makes changes to the ingredients. The standard recipe information I1 includes, for example, information regarding the food (food name), information regarding ingredients used to prepare the food (ingredient names), information regarding ingredient form, information regarding ingredient quantity (quantities used), information regarding the sensory evaluations of the ingredients, and information regarding chemical components contained in the ingredients. The standard recipe information I1 may also include information indicating whether or not each ingredient may be changed to other ingredients, namely ingredient customizability information. The ingredients included in the standard recipe information I1 are defined as standard ingredients according to the present embodiment. FIG. 7 specifically illustrates the standard recipe information I1 regarding "fresh pasta". The standard recipe information I1 regarding "fresh pasta" includes five standard ingredients. The five standard ingredients are more specifically: wheat flour (high-protein flour), olive oil, eggs (egg whites), eggs (egg yolks), and salt.

Standard ingredient information, which is information regarding the standard ingredients, includes information regarding the ingredients (ingredient names), information regarding ingredient form, information regarding ingredient quantity (quantities used), information regarding the sensory evaluations of the ingredients, and information regarding chemical components contained in the ingredients. The standard ingredient information may also include information indicating whether or not each standard ingredient may be changed to another ingredient, namely ingredient customizability information. FIG. 7 includes standard ingredient information "wheat flour (high-protein flour)", standard ingredient information "olive oil", standard ingredient information "eggs (egg whites)", standard ingredient information "eggs (egg yolks)", and standard ingredient information "salt". That is, the standard recipe information I1 includes pieces of standard ingredient information.

The database 90 generates the standard recipe information I1 on the basis of the recipe information and the reference ingredient information ir and transmits the standard recipe information I1 to the server 50. Note that the database 90 generates the standard recipe information I1 in this example; however, what generates the standard recipe information I1 is not limited thereto, and the server 50 may generate the standard recipe information I1. In that case, the server 50 generates the standard recipe information I1 on the basis of the recipe information corresponding to the food information stored in the database 90 and the reference ingredient information ir.

The server 50 transmits the standard recipe information I1 received from the database 90 or the standard recipe information I1 generated by the server 50 itself to the terminal device 10.

The control unit 15 included in the terminal device 10 generates candidate information Cq on the basis of the standard recipe information I1 transmitted from the server 50 and causes the display device included in the output unit 12 to display the generated candidate information Cq. For example, the candidate information Cq includes the food and ingredients corresponding to the food included in the standard recipe information I1 and one or more sensory evaluations corresponding to the ingredients. The terminal device 10 accepts a change request for the displayed candidate information Cq from the user.

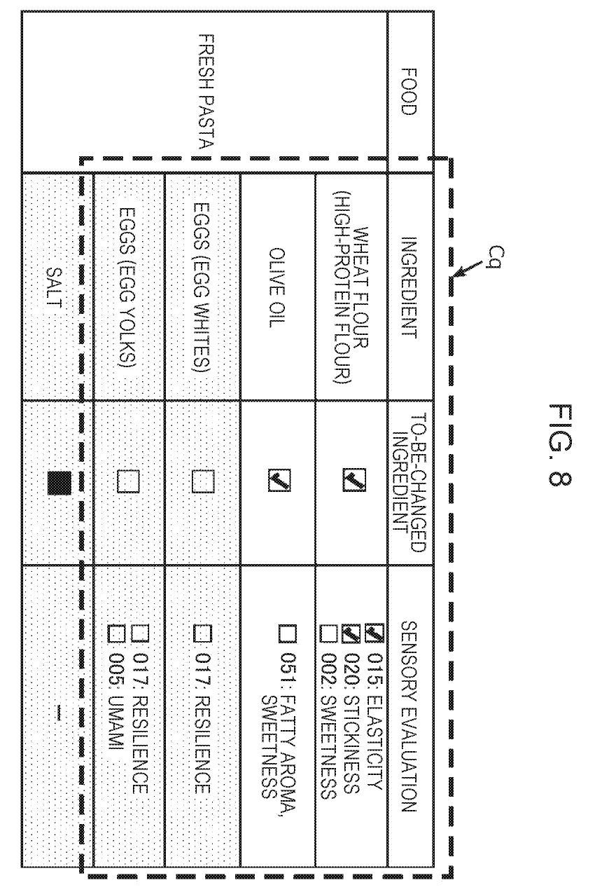

FIG. 8 is a diagram illustrating an example of the candidate information Cq listing candidates for change for the standard recipe. In FIG. 8, the candidate information Cq is illustrated as being surrounded by a thick dashed line.

The control unit 15 refers to the ingredient customizability information included in each of the pieces of standard ingredient information included in the standard recipe information I1, and generates the candidate information Cq on the basis of information corresponding to the standard ingredient information in which the ingredient customizability information has a value of "Yes". That is, the candidate information Cq does not include information corresponding to the standard ingredient information in which the ingredient customizability information has a value of "No". Specifically, among the pieces of standard ingredient information illustrated in FIG. 7, the ingredient customizability information regarding the standard ingredient information "salt" has a value of "No". Thus, the candidate information Cq illustrated in FIG. 8 does not include information corresponding to the standard ingredient information "salt".

The candidate information Cq illustrated in FIG. 8 includes information regarding the food, information regarding the ingredients, and information regarding the sensory evaluations. The information regarding the ingredients illustrated in FIG. 8 is standard ingredient information in which the ingredient customizability information has a value of "Yes". Note that, in FIG. 8, the ingredients that are not selected as to-be-changed ingredients are indicated by shaded hatching. Among the ingredients and sensory evaluations listed in the candidate information Cq, the terminal device 10 accepts to-be-changed ingredients, which are to be changed, and sensory evaluations to be maintained from the user.

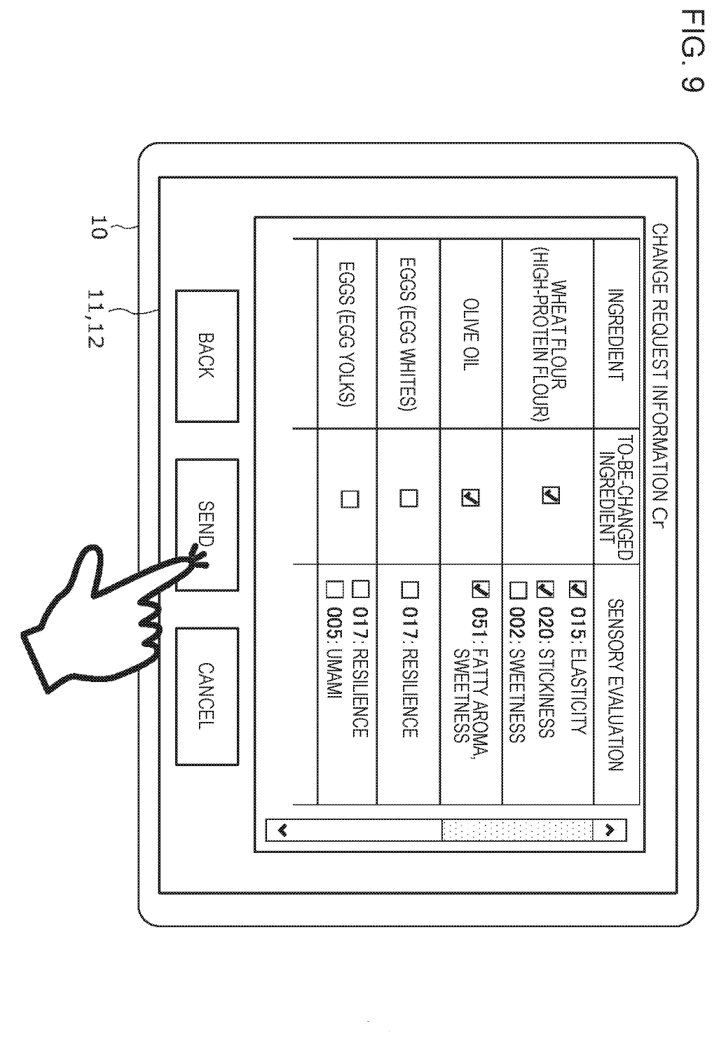

FIG. 9 is a diagram illustrating an example of a screen on which change request information Cr is displayed by the output unit (a display unit) of the terminal device 10, the change request information Cr being information reflecting the user's request for the candidate information Cq in FIG. 8.

In this example, among the standard ingredients of fresh pasta, "wheat flour" and "olive oil" are input as to-be-changed ingredients. In this example, among the sensory evaluations "elasticity, stickiness, sweetness" that "wheat flour" has, "elasticity, stickiness" are selected as sensory evaluations to be maintained. The user's intention indicates that "sweetness" may be or does not have to be maintained. The sensory evaluation "fatty aroma and sweetness" that the standard ingredient "olive oil" has is selected as a sensory evaluation to be maintained. The terminal device 10 transmits the change request information Cr to the server 50. FIG. 9 illustrates specific examples of the to-be-changed ingredients and the sensory evaluations to be maintained; however, inputs for ingredients that are not allowed to be changed or for sensory evaluations that can be changed may be accepted from the user.

The server 50 acquires the change request information Cr transmitted from the terminal device 10, and generates a chemical component list Lc on the basis of the change request information Cr.

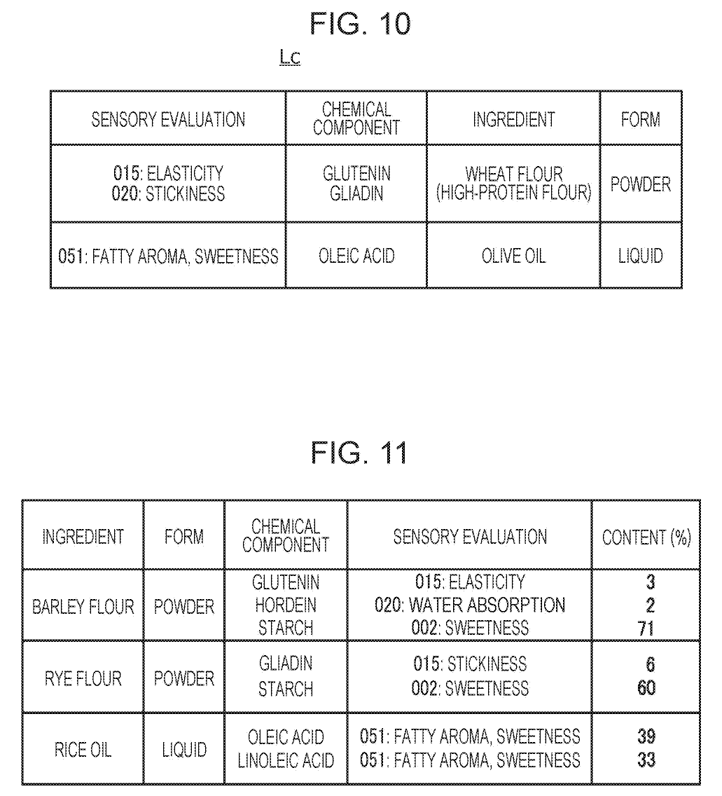

FIG. 10 is a diagram illustrating an example of the chemical component list Lc generated for the ingredients on the basis of the change request information Cr.

The chemical component list Lc includes information regarding the sensory evaluations indicated in the change request information Cr, information regarding the ingredients to be changed, and information regarding the forms of the ingredients. The chemical component list Lc also includes information regarding the above-mentioned one or more predetermined chemical components.

The server 50 generates the chemical component list Lc on the basis of the ingredients to be changed and sensory evaluations to be maintained, which are indicated in the change request information Cr, and the above-mentioned standard recipe information I1. For example, the server 50 lists chemical components so that the chemical components contained in the ingredients to be changed and the chemical components related to the sensory evaluations to be maintained match.

In this example, the chemical component "glutenin" is listed, which is a chemical component contained in "wheat flour" and related to "elasticity". The chemical component "gliadin" is also listed, which is a chemical component contained in "wheat flour" and related to "stickiness". The chemical component "oleic acid" is also listed, which is a chemical component contained in "olive oil" and related to "fatty aroma and sweetness". The reason for focusing on chemical components is to find ingredients serving as replacements that have the characteristics of the ingredients to be changed.

The server 50 transmits the generated chemical component list Lc to the database 90. The database 90 generates, from the ingredient database 92, information regarding substitute ingredients serving as replacements for the ingredients to be changed. Specifically, the database 90 extracts ingredients containing the chemical components in the chemical component list Lc as substitute ingredient candidates from the reference ingredient information ir in the ingredient database 92.

FIG. 11 is a diagram illustrating an example of substitute ingredient candidates serving as possible replacements for the standard ingredients.

In FIG. 11, ingredients that have the same chemical components and the same sensory evaluations as the ingredients to be changed are extracted as substitute ingredient candidates. In this example, the substitute ingredient candidates for "wheat flour" are "barley flour" and "rye flour". "Barley flour" has the same chemical component "glutenin" and the same sensory evaluation "elasticity" as "wheat flour" and is thus listed as a substitute ingredient candidate for "wheat flour". In addition, "rye flour" has the same chemical component "gliadin" and the same sensory evaluation "stickiness" as "wheat flour" and is thus listed as a substitute ingredient candidate for "wheat flour".

In this example, a substitute ingredient candidate for "olive oil" is "rice oil". "Rice oil" has the same chemical component "oleic acid" and the same sensory evaluation "fatty aroma and sweetness" as "olive oil" and is thus listed as the substitute ingredient candidate for "olive oil".

Note that the above-mentioned description illustrates the example in which the substitute ingredients are extracted so that the predetermined chemical components are matched; however, the way in which the substitute ingredients are extracted is not limited to this. For example, the database 90 may extract candidates for substitute ingredients so that the forms of the ingredients to be changed match those of the substitute ingredients. The database 90 may also extract, as candidates for the substitute ingredients, reference ingredients that contain sensory evaluations associated with standard ingredients among the reference ingredients.

The above-mentioned description illustrates the example in which the database 90 generates substitute ingredient candidates; however, what generates substitute ingredient candidates is not limited thereto, and substitute ingredient candidates may be generated by the server 50. In that case, the server 50 acquires, from the database 90, the reference ingredient information ir corresponding to the chemical components in the chemical component list Lc, and generates and acquires information regarding substitute ingredient candidates on the basis of this reference ingredient information ir. The server 50 generates substitute recipe information I2 including substitute ingredient information i2 on the basis of the substitute ingredient candidates.

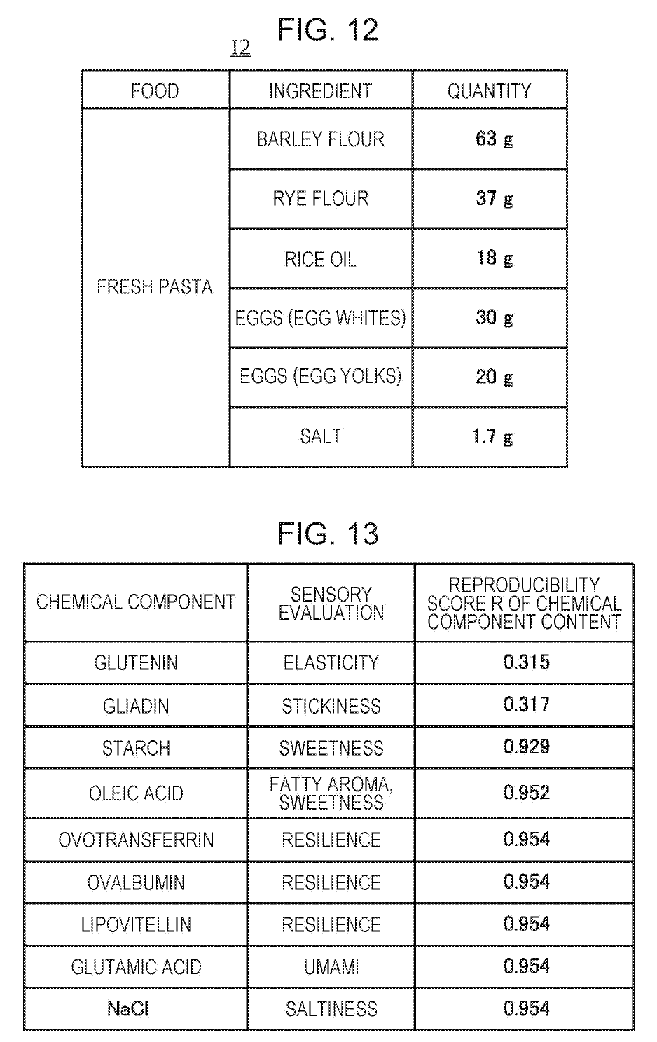

FIG. 12 is a diagram illustrating an example of the substitute recipe information I2 including the substitute ingredient information i2.

The substitute recipe information I2 is information indicating a substitute recipe for the food subject to the recipe change. The substitute recipe information I2 includes information regarding the food subject to the recipe change and information regarding substitute ingredients needed to prepare the food. FIG. 12 illustrates substitute recipe information I2 regarding "fresh pasta".

The substitute ingredient information i2, which is information regarding substitute ingredients, includes information regarding ingredients to be substitute ingredients, the forms of the substitute ingredients, the quantities of the substitute ingredients, the sensory evaluations of the substitute ingredients, and chemical components contained in the substitute ingredients.

FIG. 12 illustrates the substitute ingredient information i2 regarding the ingredients "barley flour, rye flour, rice oil, eggs (egg whites), eggs (egg yolks), salt" of a substitute recipe for "fresh pasta". Note that since the food subject to the recipe change is prepared using at least one substitute ingredient, the substitute recipe includes the same ingredients as and different ingredients from those included in the standard recipe information I1.

In this manner, the server 50 derives, as at least one substitute ingredient, one or more reference ingredients that contain chemical components contained in the standard ingredients from the reference ingredients in the ingredient database 92. For example, the server 50 derives at least one substitute ingredient from the reference ingredients so as to contain all of the one or more predetermined chemical components contained in the standard ingredients. In this example, the predetermined chemical components contained in the standard ingredient "wheat flour" are "glutenin" and "gliadin", and the chemical components contained in the substitute ingredients "barley flour" and "rye flour" are "glutenin" and "gliadin", respectively.

Note that the server 50 may also derive at least one substitute ingredient from the reference ingredients so as to include not only the chemical components but also all of one or more predetermined sensory evaluations associated with the standard ingredients. In this example, the predetermined sensory evaluations related to the standard ingredient "wheat flour" are "elasticity" and "stickiness", and the predetermined sensory evaluations associated with the substitute ingredients "barley flour" and "rye flour" are "elasticity" and "stickiness", respectively.

Furthermore, the server 50 also derives the quantities of the substitute ingredients.

For example, the quantities of the substitute ingredients are calculated so that the ratios of the chemical components are as close as possible to those of the standard ingredients. In this case, in "wheat flour", the content of "glutenin" is 6% and the content of "gliadin" is 7% (see FIG. 6). In contrast, the content of "glutenin" in "barley flour" is 3%, and the content of "gliadin" in "rye flour" is 6% (see FIG. 11). Thus, if the ratio of "glutenin" to "gliadin" contained in the substitute ingredients is to be 6:7 as in "wheat flour", the ratio of "barley flour" to "rye flour" needs to be 63:37. For example, in a case where the quantity of "wheat flour" to be changed is 100 g, the substitute ingredients "barley flour" and "rye flour" are prepared so that the total quantity is 100 g. As illustrated in FIG. 12, the quantity of "barley flour" is 63 g, and the quantity of "rye flour" is 37 g.

In this manner, the server 50 derives the quantities of substitute ingredients on the basis of the quantity of a standard ingredient, the contents of the chemical components contained in the standard ingredient, and the content of the chemical components contained in the substitute ingredients. For example, the server 50 derives the quantities of substitute ingredients on the basis of the quantity of a standard ingredient, the contents of one or more predetermined chemical components contained in the standard ingredient, and the contents of the one or more predetermined chemical components contained in the substitute ingredients. This makes it possible to prepare the food or a food equivalent thereto without using one or more of the ingredients needed to prepare the food.

Note that one substitute ingredient candidate or a set of substitute ingredient candidates is indicated in FIG. 11 mentioned above; however, substitute ingredient candidates are not limited thereto. As substitute ingredient candidates, the server 50 may extract substitute ingredient candidates or sets of substitute ingredient candidates and select one substitute ingredient or a set of substitute ingredients from the substitute ingredient candidates.

The relationship between the type of standard ingredient and the type of substitute ingredient does not have to be a one-to-one relationship, as with olive oil, and may be a one-to-many relationship, as with wheat flour. In addition, the relationship between the type of standard ingredient and the type of substitute ingredient does not have to be a one-to-one relationship and may be a many-to-one relationship.

For example, the server 50 may derive, for substitute ingredient candidates, reproducibility scores R indicating the degrees of reproducibility of the standard ingredients and generate a substitute recipe using the substitute ingredients with high reproducibility scores R. Each reproducibility score R is the ratio of a value obtained based on the quantity of the substitute ingredient and the content of the chemical component contained in the substitute ingredient to a value obtained based on the quantity of the standard ingredient and the content of the chemical component contained in the standard ingredient. The reproducibility score R is expressed as a numerical value greater than or equal to 0 and less than or equal to 1. The closer the numerical value is to 1, the higher the reproducibility score R becomes.

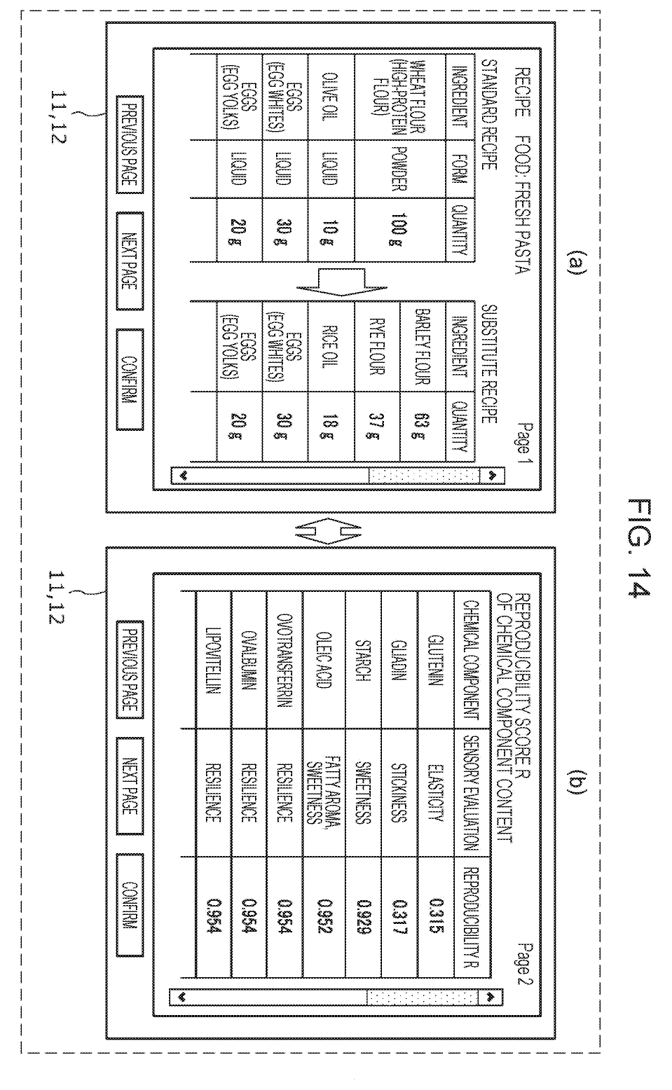

FIG. 13 is a diagram illustrating the reproducibility scores R of the contents of the chemical components in the substitute ingredients relative to the standard ingredients. Note that FIG. 13 also lists the sensory evaluations.

The reproducibility score R of the content of "glutenin" is calculated using, as the denominator, the value obtained by multiplying the quantity of "wheat flour" (100 g) by the content of "glutenin" (6%) in the wheat flour in the standard ingredients and using, as the numerator, the value obtained by multiplying the quantity of "barley flour" (63 g) in the substitute ingredients by the content of "glutenin" (3%) in the barley flour. The reproducibility score R of the content of "glutenin" is R = (63×0.03)/(100×0.06) = 0.315.

The reproducibility score R of the content of "gliadin" is calculated using, as the denominator, the value obtained by multiplying the quantity of "wheat flour" (100 g) in the standard ingredients by the content of "gliadin" (7%) in the wheat flour and using, as the numerator, the value obtained by multiplying the quantity of "rye flour" (37 g) in the substitute ingredients by the content of "gliadin" (6%) in the rye flour. The reproducibility score R of the content of "gliadin" is R = (37×0.06)/(100×0.07) = 0.317.

The reproducibility score R of the content of "starch" is calculated using, as the denominator, the value obtained by multiplying the quantity of "wheat flour" (100 g) in the standard ingredients by the content of "starch" (72%) in the wheat flour and using, as the numerator, the sum of the value obtained by multiplying the quantity of "barley flour" (63 g) in the substitute ingredients by the content of "starch" (71%) in the barley flour and the value obtained by multiplying the quantity of "rye flour" (37 g) in the substitute ingredients by the content of "starch" (60%) in the rye flour. The reproducibility score R of the content of "starch" is R = ((63×0.71) + (37×0.60))/(100×0.72) = 0.929. In the example of "starch", since there are two substitute ingredients that have the same chemical component, the reproducibility score R is the sum of two reproducibility scores R obtained for the two respective substitute ingredients.

The server 50 calculates the reproducibility scores R as mentioned above, extracts substitute ingredient with high reproducibility scores R from the substitute ingredient candidates, and generates substitute recipe information I2 including the extracted substitute ingredients. The server 50 transmits the generated substitute recipe information I2 to the terminal device 10.

FIG. 14 includes diagrams illustrating examples of screens displaying the substitute ingredients for the standard ingredients and the reproducibility scores R of the contents of the chemical components.

FIG. 14 (a) lists the ingredients for the standard recipe and the ingredients for a substitute recipe. FIG. 14 (a) also lists the forms and quantities of the ingredients.

FIG. 14 (b) lists the chemical components contained in the ingredients, the sensory evaluations corresponding to the chemical components, and the reproducibility scores R of the contents of the chemical components. According to FIG. 14 (b), the degrees of reproducibility of the contents of the chemical components in the substitute recipe corresponding to the standard recipe can be listed numerically. Note that the reproducibility scores R may be included in the substitute recipe information I2.

As described above, the information provision system 1 according to the present embodiment includes the server 50, which derives substitute ingredients serving as replacements for the standard ingredients. The server 50 acquires information indicating the standard ingredients and the quantities of the standard ingredients and derives at least one substitute ingredient from reference ingredients and information indicating the quantity of the at least one substitute ingredient on the basis of the quantities of the standard ingredients, the contents of the chemical components contained in the standard ingredients, and the contents of the chemical components contained in each of the reference ingredients.

In this manner, even if one or more of the ingredients needed to prepare the food are not used, the food or a food equivalent thereto can be prepared by deriving substitute ingredients and the quantities of the substitute ingredients on the basis of the quantities of the standard ingredients, the contents of the chemical components contained in the standard ingredients, and the contents of the chemical components contained in each of the reference ingredients.

Note that, in the above-mentioned description, the example has been described in which data corresponding to the change request information Cr is stored in the database 90; however, there may be a case where the data corresponding to the change request information Cr is not stored in the database 90. Regarding the above-mentioned description, a case where information regarding "rice oil" and information regarding "oleic acid" are not stored in the ingredient database 92 will be described as an example.

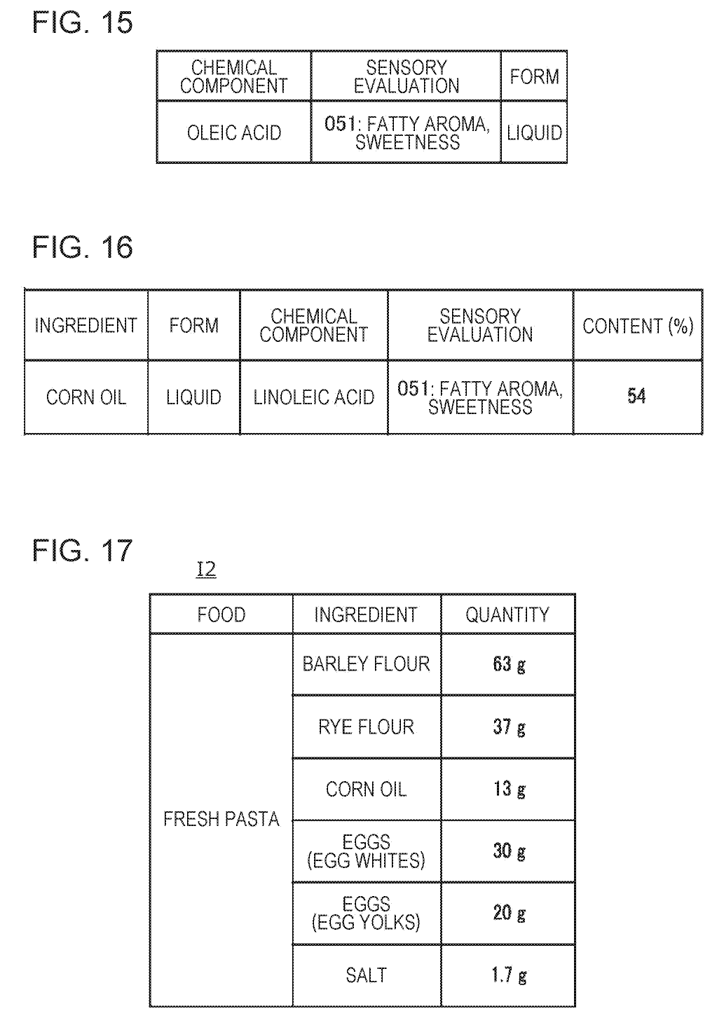

FIG. 15 is a diagram illustrating an example of ingredient information that is not stored in the database 90.

This example indicates that "oleic acid", which is a chemical component contained in "rice oil", and information regarding a sensory evaluation of "fatty aroma and sweetness" corresponding to "oleic acid" are not present in the database 90.

In a case where no substitute ingredient candidate has a matching chemical component, the information provision system 1 extracts other substitute ingredients on the basis of the sensory evaluations corresponding to the chemical components and the forms of the ingredients.

FIG. 16 is a diagram illustrating an example of other substitute ingredient candidates.

First, the server 50 obtains chemical components corresponding to the sensory evaluation "fatty aroma and sweetness", which is the same as that for "oleic acid", and transmits the chemical components to the database 90. The database 90 extracts, as a substitute ingredient candidate, an ingredient having the chemical component and having the same form as "rice oil" from the reference ingredient information ir. This example indicates that the ingredient form is "liquid" and the chemical component having the sensory evaluation "fatty aroma and sweetness" is "linoleic acid" and that the ingredient satisfying these is "corn oil". The database 90 transmits the extracted substitute ingredient candidate to the server 50.

In this manner, the information provision system 1 may derive the substitute ingredient information i2 by deriving, as at least one substitute ingredient, a reference ingredient including a sensory evaluation associated with the standard ingredient among the reference ingredients. Furthermore, the information provision system 1 may derive at least one substitute ingredient from the reference ingredients so as to include all of one or more predetermined sensory evaluations associated with the standard ingredient.

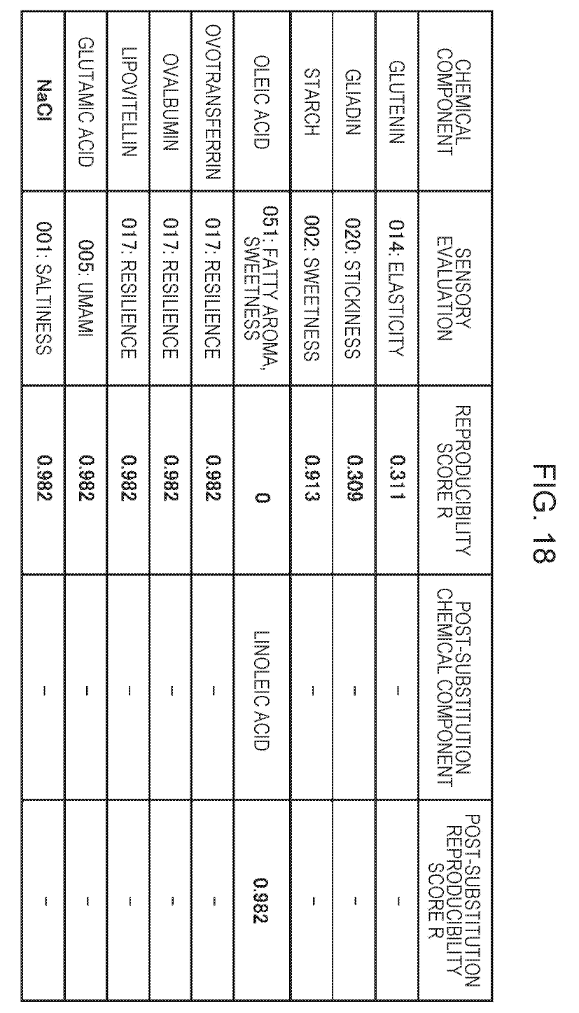

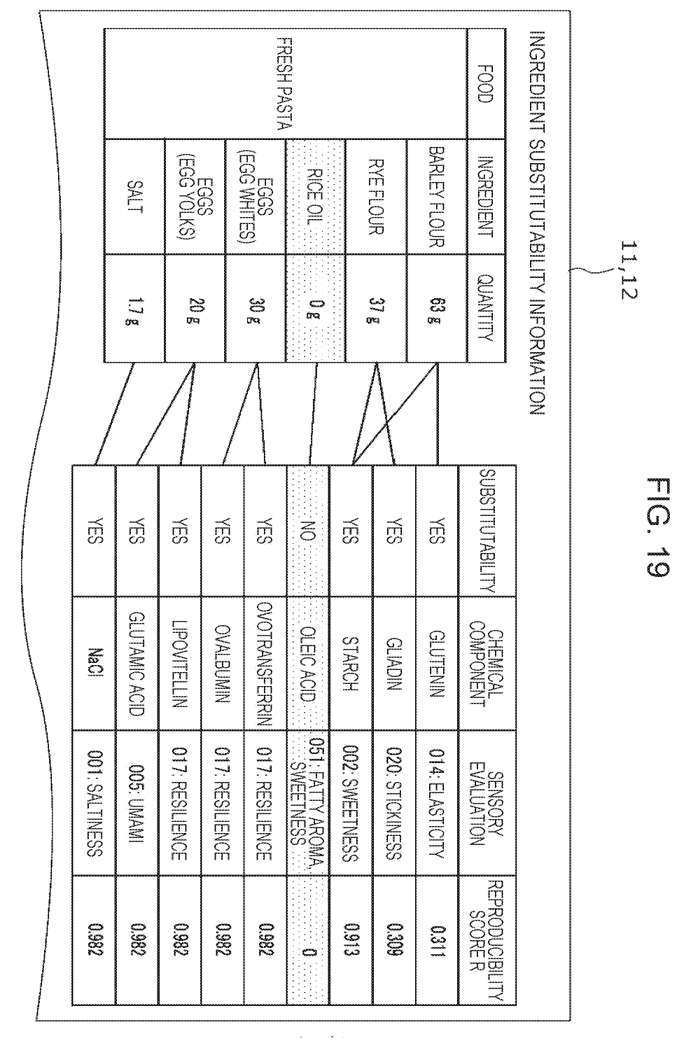

FIG. 17 is a diagram illustrating an example of the substitute recipe information I2 using other substitute ingredients.

FIG. 17 illustrates an example in which one of the substitute ingredients for "fresh pasta" is "corn oil" instead of "rice oil". The server 50 determines the quantities of post-substitution ingredients such that the contents of the chemical components contained in the pre-substitution ingredients and those contained in the post-substitution ingredients are as equal as possible. In this example, the server 50 determines the quantity of "corn oil" such that the content of the chemical component "oleic acid" contained in "rice oil" before substitution and the content of the chemical component "linoleic acid" contained in "corn oil" after substitution are equal.

FIG. 18 is a diagram illustrating information regarding the other substitute ingredients.

As the information regarding the other substitute ingredients, FIG. 18 illustrates chemical component, sensory evaluation, the reproducibility score R of chemical component content for pre-substitution ingredients, and chemical component and the reproducibility score R of chemical component content for post-substitution ingredients. This example indicates that a chemical component in a pre-substitution ingredient is "oleic acid", its sensory evaluation is "fatty aroma and sweetness", and the above-mentioned reproducibility score R is 0% and that a post-substitution chemical component is "linoleic acid" and the above-mentioned reproducibility score R is 0.982. According to this, the chemical component in the substitute ingredient and the degree of reproducibility of the chemical component content can be clearly indicated.

The example in which a substitute ingredient was found has been described above, but there may be a case where no substitute ingredient is found. Regarding the above-mentioned description, a case where information regarding the ingredient "rice oil", the chemical component "oleic acid", the sensory evaluation "fatty aroma and sweetness", and the ingredient form "liquid" is not stored in the ingredient database 92 will be described as an example.

FIG. 19 is a diagram illustrating an example of a screen displayed in a case where no substitute ingredient is available.

In a case where the information provision system 1 could not find other substitute ingredient candidates, the information provision system 1 outputs information indicating that no substitute ingredient was found. FIG. 19 illustrates that no information regarding "rice oil" or "corn oil" is stored in the ingredient database 92, and no ingredient with the sensory evaluation "fatty aroma and sweetness" and in the form of "liquid" was found. In a case where no substitute ingredient was found, the reproducibility score R of the content of the chemical component is expressed as 0. This would clearly indicate that no substitute ingredient was found.

### Operation of Information Provision System

The operation of the information provision system 1 according to the embodiment will be described.

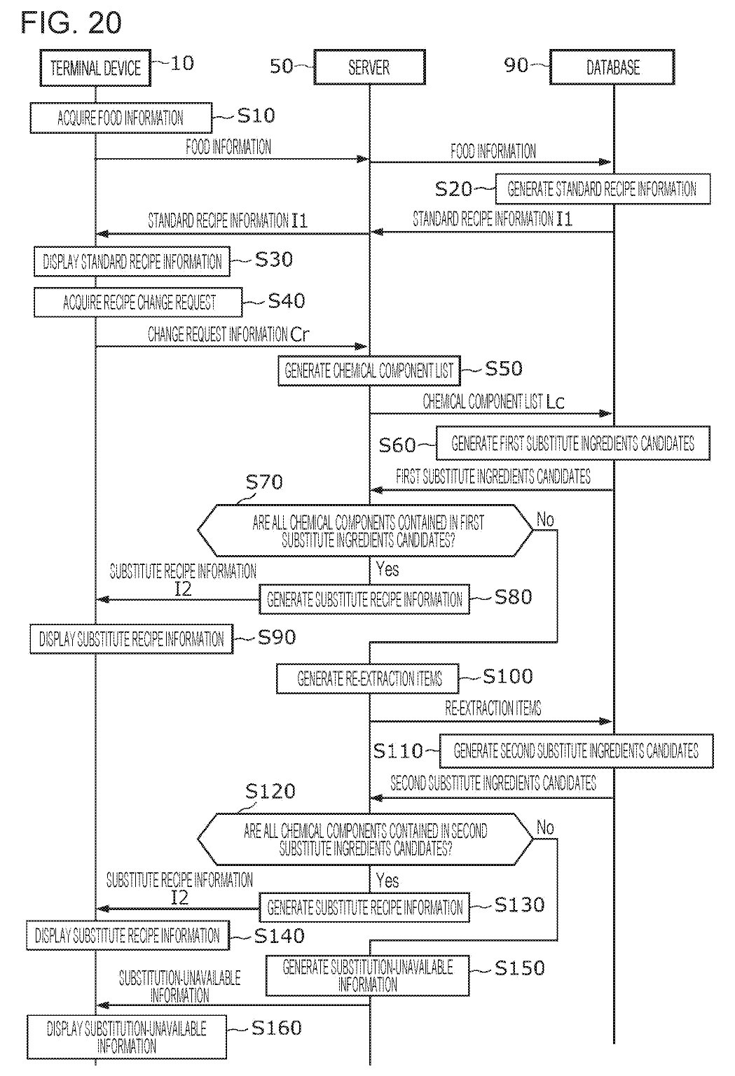

FIG. 20 is a diagram illustrating an operation flowchart of the information provision system 1.

As illustrated in FIG. 20, the terminal device 10 acquires food information from the user (Step S10). The food information is information regarding the food whose recipe is about to be changed. The terminal device 10 outputs the acquired food information to the server 50.

The server 50 acquires the food information output from the terminal device 10, and outputs this food information to the database 90.

The database 90 generates standard recipe information I1 on the basis of the food information (Step S20).

The standard recipe information I1 is information regarding the original recipe for the food whose recipe is about to be changed. The standard recipe information I1 includes information regarding the food subject to the recipe change and information regarding standard ingredients, which are the original ingredients needed to prepare the food. Standard ingredient information i1 regarding the standard ingredients includes information regarding ingredients serving as the standard ingredients, the forms of the standard ingredients, the quantities of the standard ingredients, the sensory evaluations of the standard ingredients, and chemical components contained in the standard ingredients. The standard ingredient information i1 also includes information regarding the customizability of each ingredient when changing the ingredient.

The database 90 outputs the standard recipe information I1 to the server 50. The server 50 acquires the standard recipe information I1 output from the database 90 and outputs the standard recipe information I1 to the terminal device 10.

The terminal device 10 displays the standard recipe information I1 output from the server 50 (Step S30). For example, the terminal device 10 displays information regarding the ingredients, the customizability indicating whether or not the ingredients can be changed to other ingredients, and the sensory evaluations associated with the ingredients, the information being represented as the standard recipe.

The terminal device 10 that displays the standard recipe information I1 acquires a change request for the standard recipe from the user (Step S40). For example, among the ingredients and sensory evaluations listed in the standard recipe, the terminal device 10 acquires information regarding to-be-changed ingredients, which are to be changed, and sensory evaluations that are to be maintained without change, from the user. The terminal device 10 outputs, to the server 50, change request information Cr input by the user.

The server 50 acquires the change request information Cr output from the terminal device 10, and generates a chemical component list Lc on the basis of the change request information Cr (Step S50).

The chemical component list Lc includes information regarding the sensory evaluations and the ingredients to be changed, which are indicated in the change request information Cr, and the forms of the ingredients. The chemical component list Lc also includes information regarding one or more predetermined chemical components. The server 50 generates the chemical component list Lc on the basis of the ingredients to be changed and sensory evaluations to be maintained, which are indicated in the change request information Cr, and the standard recipe information I1. For example, the server 50 lists the chemical components so that the chemical components contained in the ingredients to be changed and the chemical components related to the sensory evaluations to be maintained match. The server 50 outputs the generated chemical component list Lc to the database 90.

The database 90 generates, from the ingredient database 92, first substitute ingredient candidates serving as possible replacements for the ingredients to be changed (Step S60). Specifically, the database 90 extracts ingredients containing the chemical components in the chemical component list Lc as the first substitute ingredient candidates from the reference ingredient information ir in the ingredient database 92. The substitute ingredient candidates illustrated in FIG. 11 are examples of the first substitute ingredient candidates. The number of first substitute ingredient candidates may be one or more. The database 90 outputs the extracted first substitute ingredient candidates to the server 50.

The server 50 determines whether or not all of predetermined chemical components contained in the standard ingredients are contained in the first substitute ingredient candidates (Step S70).

In a case where all the chemical components are contained in the first substitute ingredient candidates (Yes in S70), the server 50 extracts substitute ingredients that contain all the chemical components to generate substitute recipe information I2 that includes the substitute ingredients (Step S80).

The substitute recipe information I2 is information indicating a substitute recipe for the food subject to the recipe change. The substitute recipe information I2 includes information regarding the food subject to the recipe change and information regarding substitute ingredients needed to prepare the food. Substitute ingredient information i2, which is information regarding substitute ingredients, includes information regarding ingredients to be substitute ingredients, the forms of the substitute ingredients, the quantities of the substitute ingredients, the sensory evaluations of the substitute ingredients, and chemical components contained in the substitute ingredients.

The substitute recipe information I2 may also include the reproducibility scores R of the contents of the chemical components indicating the degrees of reproducibility of the substitute ingredients relative to the standard ingredients. The server 50 outputs the substitute recipe information I2 to the terminal device 10.

The terminal device 10 displays the substitute recipe information I2 output from the server 50 (Step S90). As a result, the terminal device 10 presents the user with information regarding the substitute ingredients serving as replacements for the standard ingredients.

In contrast, in a case where not all the chemical components are contained in the first substitute ingredient candidates (No in S70), the server 50 generates re-extraction items that are items for re-extracting substitute ingredient candidates (Step S100). The re-extraction items include information regarding the sensory evaluations and ingredient forms related to chemical components that were not present in the first substitute ingredient candidates. The server 50 outputs the re-extraction items to the database 90.

The database 90 generates second substitute ingredient candidates on the basis of the re-extraction items output from the server 50. Specifically, the database 90 extracts, as substitute ingredients, reference ingredients that satisfy the sensory evaluations and ingredient forms included in the re-extraction items to generate the second substitute ingredient candidates (Step S110). The other substitute ingredient candidate illustrated in FIG. 16 is one example of the second substitute ingredient candidates. The database 90 outputs the second substitute ingredient candidates to the server 50.

The server 50 determines whether or not all of the predetermined chemical components contained in the standard ingredients are contained in the second substitute ingredient candidates (Step S120).

In a case where all the chemical components are contained in the second substitute ingredient candidates (Yes in S120), the server 50 extracts substitute ingredients that contain all the chemical components to generate substitute recipe information I2 that includes the substitute ingredients (Step S130). This substitute recipe information I2 includes information regarding the chemical components in the substitute ingredients and the reproducibility scores R of the contents of the chemical components.

The terminal device 10 displays the substitute recipe information I2 output from the server 50 (Step S140). As a result, the terminal device 10 presents the user with information regarding the substitute ingredients serving as replacements for the standard ingredients.

In contrast, in a case where not all the chemical components are contained in the second substitute ingredient candidates (No in S120), the server 50 generates substitution-unavailable information indicating that no substitute ingredient was found (S150). The substitution-unavailable information includes information regarding chemical components and sensory evaluations that are not substitutable. The server 50 outputs the substitution-unavailable information to the terminal device 10.

The terminal device 10 displays the substitution-unavailable information output from the server 50 (Step S160). As a result, the terminal device 10 presents the user with information indicating that no substitute ingredients serving as replacements for the standard ingredients was found.

In this manner, an information provision method according to the present embodiment is an information provision method including: acquiring standard ingredient information i1 indicating a standard ingredient and a quantity of the standard ingredient, deriving substitute ingredient information i2 indicating at least one substitute ingredient serving as a replacement for the standard ingredient from reference ingredients and a quantity of the at least one substitute ingredient, based on the quantity of the standard ingredient, a content of a chemical component contained in the standard ingredient, and a content of a chemical component contained in each of the reference ingredients, and outputting the substitute ingredient information i2.

In this manner, even if one or more of the ingredients needed to prepare a food are not used, the food or a food equivalent thereto can be prepared by deriving a substitute ingredient and the quantity of the substitute ingredient based on the quantity of a standard ingredient, the content of a chemical component contained in the standard ingredient, and the content of a chemical component contained in each of reference ingredients.

### Modification of Embodiment

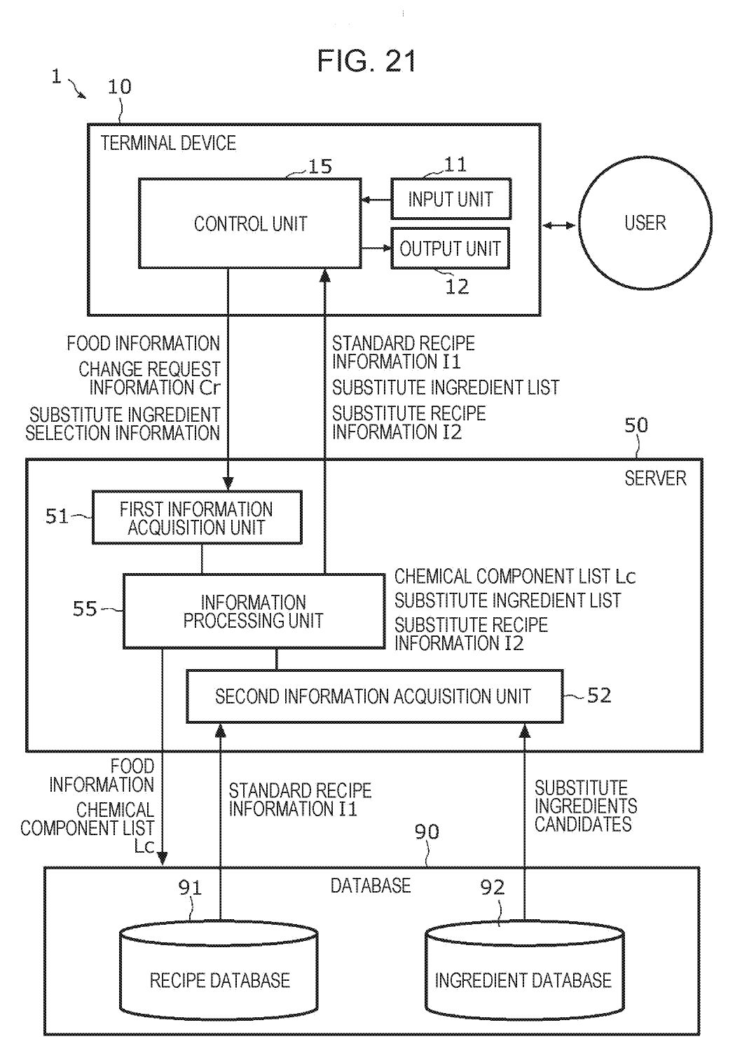

An information provision system according to a modification of the embodiment will be described with reference to FIGS. 21 to 24. In this modification, an example will be described in which the user selects substitute ingredients from a substitute ingredient list.

FIG. 21 is a block diagram illustrating a functional configuration of an information provision system 1 according to the modification of the embodiment.

The information provision system 1 according to the modification also includes the terminal device 10, the server 50, and the database 90. In the modification, the database 90 transmits substitute ingredient candidates to the server 50 on the basis of the chemical component list Lc, and the server 50 transmits a substitute ingredient list including the substitute ingredient candidates to the terminal device 10. The terminal device 10 accepts an input to select substitute ingredients from the substitute ingredient list and transmits the input to the server 50. The server 50 generates substitute recipe information I2 on the basis of information regarding the selected substitute ingredients and transmits the substitute recipe information I2 to the terminal device 10.

In the following, the configuration of the information provision system 1 according to the modification will be described in detail, while describing an operation flowchart of the information provision system 1.

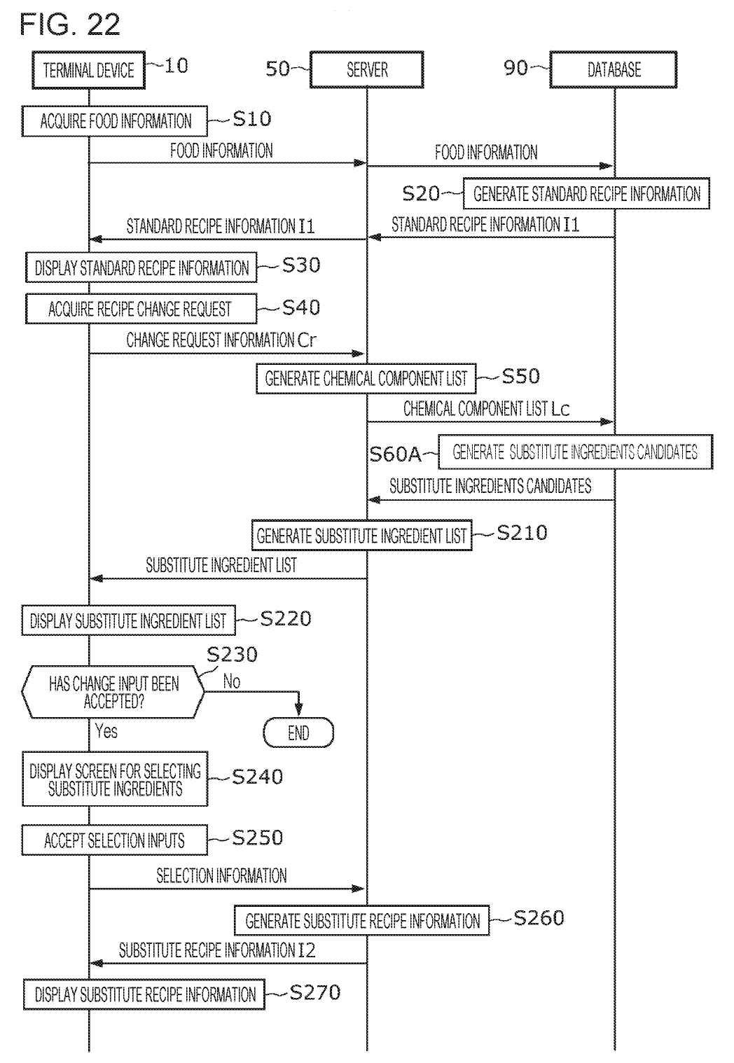

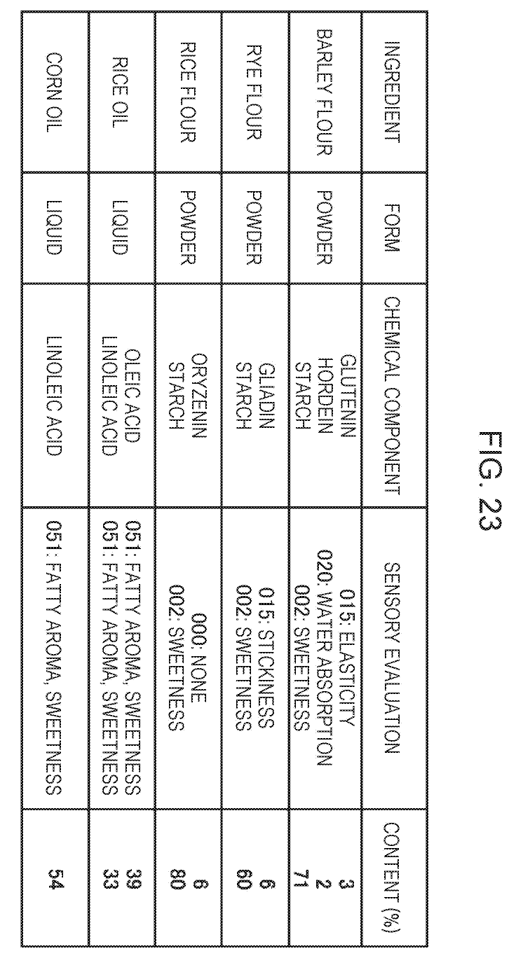

FIG. 22 is a diagram illustrating the operation flowchart of the information provision system according to the modification of the embodiment.

Steps S10 to S50 illustrated in FIG. 22 are substantially the same as those in FIG. 20 according to the embodiment. As illustrated in FIG. 22, the database 90 generates substitute ingredient candidates on the basis of the chemical component list Lc (Step S60A) and outputs the substitute ingredient candidates to the server 50.

The server 50 generates a substitute ingredient list including the substitute ingredient candidates (Step S210).

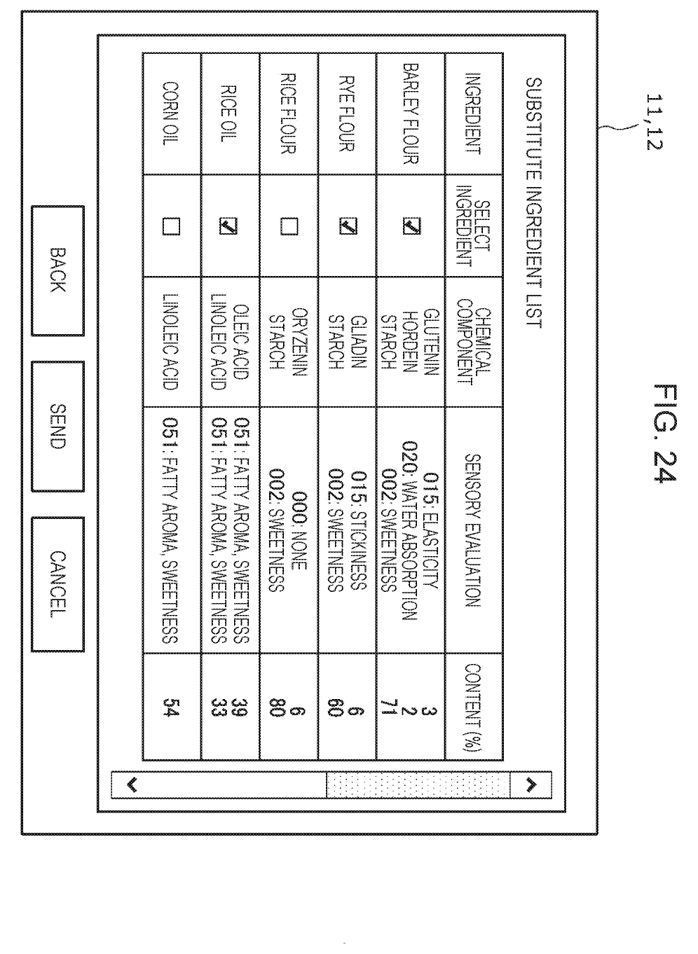

FIG. 23 is a diagram illustrating an example of the substitute ingredient list including the substitute ingredient candidates.

The substitute ingredient list includes ingredients usable as substitutes. Examples of the ingredients usable as substitutes include ingredients having the same chemical components as pre-substitution ingredients, and ingredients having the same sensory evaluations and the same forms as the pre-substitution ingredients. FIG. 23 lists, as the ingredients usable as substitutes, ingredients about "barley flour, rye flour, rice flour, rice oil, corn oil". This example does not list one or more substitute ingredients derived through arithmetic processing as in the embodiment but indicates a state in which ingredients prior to derivation are listed in a duplicated manner. For example, two ingredients "rice oil, corn oil" are listed as ingredients serving as replacements for "olive oil". The server 50 outputs the substitute ingredient list including the substitute ingredient candidates to the terminal device 10.

The terminal device 10 acquires the substitute ingredient candidates output from the server 50, and displays the substitute ingredient list including substitute ingredients (Step S220).

The terminal device 10 determines whether or not to change substitute ingredients in the substitute ingredient list. Specifically, the terminal device 10 determines whether or not a change input has been accepted for substitute ingredients (Step S230).

In a case where no change input has been accepted (No in S230), this flowchart ends. In contrast, in a case where a change input has been accepted (Yes in S230), the terminal device 10 displays a screen for selecting substitute ingredients (Step S240).

FIG. 24 is a diagram illustrating an example of a screen for selecting substitute ingredients from the substitute ingredient list.

The terminal device 10 illustrated in FIG. 24 allows the user to select their desired substitute ingredients from the substitute ingredient list.

The terminal device 10 accepts an input for selecting substitute ingredients (Step S250). In this example, "barley flour, rye flour, rice oil" are the substitute ingredients desired by the user. The terminal device 10 outputs, to the server 50, substitute-ingredient selection information including information regarding the selected substitute ingredients.

The server 50 generates substitute recipe information I2 so as to include the selected substitute ingredients (Step S260) and outputs this substitute recipe information I2 to the terminal device 10. The terminal device 10 displays the substitute recipe information I2 output from the server 50 (Step S270). This allows the user to select their desired substitute ingredients.

### SUMMARY

In an information provision method according to the present disclosure, standard recipe information I1 is generated based on information regarding an ingredient subject to a recipe change. The standard recipe information I1 is generated based on recipe information and reference ingredient information regarding the ingredient subject to the recipe change, from recipe information and reference ingredient information ir stored in the database 90. Next, information regarding an ingredient to be changed in a standard recipe or regarding sensory evaluation is received from the user, and chemical components common to the ingredient to be changed and its sensory evaluation are listed. Next, substitute ingredient candidates having the above-mentioned chemical components are extracted from the reference ingredient information ir. One or more substitute ingredients are selected from the substitute ingredients to generate substitute recipe information. For example, when selecting a substitute ingredient, a substitute ingredient having a high reproducibility score R of the content of a certain chemical component may be selected. This makes it possible to provide information that enables a food or a food equivalent thereto to be prepared without using one or more of the ingredients needed to prepare the food.

### Other Embodiments

The information provision method and the like according to the embodiments of the present disclosure have been described above; however, the present disclosure is not limited to each of the embodiments. Embodiments obtained by adding various modifications which those skilled in the art can conceive of to the present embodiments and embodiments constructed by combining constituent elements from different embodiments may also be included in the scope of one or more aspects of the present disclosure, provided that the embodiments do not deviate from the gist of the present disclosure.

In the above-mentioned embodiments, the server 50 and the database 90 are separate constituent elements; however, the server 50 and the database 90 may become the same single constituent element, and the server 50 may have the same function as the database 90.

In the above-mentioned embodiments, the example has been illustrated in which the information provision system 1 is constituted by the terminal device 10, the server 50, and the database 90; however, the information provision system 1 is not limited to this. For example, in a case where the terminal device 10 has the same function as the server 50, the information provision system 1 may be constituted by the terminal device 10 and the database 90. In that case, the terminal device 10 may be provided with the functions of the first information acquisition unit 51, the second information acquisition unit 52, and the information processing unit 55.

In the above-mentioned embodiments, the example has been illustrated in which the sensory evaluations of the ingredients are displayed on the screen for accepting the change request information Cr illustrated in FIG. 9; however, the screen displayed is not limited thereto. For example, the terminal device 10 may display a screen that displays chemical components in to-be-changed ingredients, which are to be changed, and that also allows selection of chemical components to be maintained from among the chemical components. In this case, the chemical components and the sensory evaluations may be associated and displayed.

In the above-mentioned embodiments, each constituent element may be realized by executing a software program appropriate for the constituent element. Each constituent element may be realized by a program execution unit, such as a central processing unit (CPU) or a processor, reading out and executing a software program recorded on a recording medium, such as a hard disk or a semiconductor memory.

Each constituent element may be realized in hardware. Each constituent element may be a circuit (or an integrated circuit). These circuits may constitute a single circuit as a whole or may be separate circuits. Each of these circuits may be a general-purpose circuit or a dedicated circuit.

The general or specific aspects of the present disclosure may also be realized as a system, a device, a method, an integrated circuit, a computer program, or a recording medium, such as a computer-readable CD-ROM. The general or specific aspects of the present disclosure may also be realized as any combination of a system, a device, a method, an integrated circuit, a computer program, and a recording medium.

For example, the present disclosure may be realized as a data processing unit in the above-mentioned embodiments or an information processing system according to the above-mentioned embodiments. The present disclosure may also be realized as an information processing method executed by a computer, such as the information processing system according to the above-mentioned embodiments. The present disclosure may be realized as a program for causing a computer to execute such an information processing method, or as a computer-readable non-transitory recording medium in which such a program is recorded.

An information provision method according to the present disclosure can be widely used for services that provide cooking information.

What is claimed is:

1. An information provision method, the information provision method being executed by a computer, the method comprising:
acquiring standard ingredient information indicating a standard ingredient and a quantity of the standard ingredient;
deriving, from reference ingredients, substitute ingredient information indicating at least one substitute ingredient serving as a replacement for the standard ingredient and a quantity of the at least one substitute ingredient, based on the quantity of the standard ingredient, a content of a chemical component contained in the standard ingredient, and a content of a chemical component contained in each of the reference ingredients; and
outputting the substitute ingredient information.

2. The information provision method according to claim 1, wherein
in the deriving the substitute ingredient information,
a reference ingredient that contains the chemical component contained in the standard ingredient is selected, as the at least one substitute ingredient, from among the reference ingredients, and
the quantity of the at least one substitute ingredient is derived based on the quantity of the standard ingredient, the content of the chemical component contained in the standard ingredient, and the content of the chemical component contained in the at least one substitute ingredient.

3. The information provision method according to claim 1, wherein
the chemical component contained in the standard ingredient includes one or more predetermined chemical components contained in the standard ingredient, and
in the deriving the substitute ingredient information,
the at least one substitute ingredient is derived from the reference ingredients so as to contain all of the one or more predetermined chemical components contained in the standard ingredient, and
the quantity of the at least one substitute ingredient is derived based on the quantity of the standard ingredient, contents of all of the one or more predetermined chemical components contained in the standard ingredient, and contents of all of the one or more predetermined chemical components contained in the at least one substitute ingredient.

4. The information provision method according to claim 3, wherein
the at least one substitute ingredient contains a different chemical component from the one or more predetermined chemical components contained in the standard ingredient.

5. The information provision method according to claim 1, wherein
the standard ingredient information is included in standard recipe information indicating a standard recipe for a food prepared using the standard ingredient,
information indicating the content of the chemical component contained in each of the reference ingredients is included in reference ingredient information,
the substitute ingredient information is included in substitute recipe information indicating a substitute recipe for the food prepared using the at least one substitute ingredient, and
in the deriving the substitute ingredient information, the substitute recipe information is derived based on the standard recipe information and the reference ingredient information.

6. The information provision method according to claim 5, wherein
the standard ingredient information further includes information indicating a sensory evaluation associated with the standard ingredient,
the reference ingredient information further includes information indicating a sensory evaluation associated with each of the reference ingredients,
the substitute ingredient information further includes information indicating a sensory evaluation associated with the substitute ingredient, and
in the deriving the substitute ingredient information, a reference ingredient that includes the sensory evaluation associated with the standard ingredient is derived from the reference ingredients as the at least one substitute ingredient to derive the substitute ingredient information.

7. The information provision method according to claim 6, wherein
the sensory evaluation associated with the standard ingredient includes one or more predetermined sensory evaluations associated with the standard ingredient, and
in the deriving the substitute ingredient information, the at least one substitute ingredient is derived from the reference ingredients so as to include all of the one or more predetermined sensory evaluations associated with the standard ingredient.

8. The information provision method according to claim 1, wherein
in the deriving the substitute ingredient information, a reproducibility score indicating a degree of reproducibility of the standard ingredient is derived, and the at least one substitute ingredient is derived based on the reproducibility score.

9. The information provision method according to claim 8, wherein
the reproducibility score is a ratio of a value obtained based on the quantity of the substitute ingredient and the content of the chemical component contained in the substitute ingredient to a value obtained based on the quantity of the standard ingredient and the content of the chemical component contained in the standard ingredient.

10. The information provision method according to claim 8, wherein
the substitute ingredient includes substitute ingredients, and
the reproducibility score is a ratio of a sum of values obtained for each of the substitute ingredients, each value being obtained based on the quantity of the substitute ingredient and the content of the chemical component contained in the substitute ingredient, to a value obtained based on the quantity of the standard ingredient and the content of the chemical component contained in the standard ingredient.

11. The information provision method according to claim 1, wherein
in the deriving the substitute ingredient information,
substitute ingredient candidates serving as possible replacements for the standard ingredient are extracted from the reference ingredients,
selection of at least one substitute ingredient serving as a substitute for the standard ingredient from among the substitute ingredient candidates is accepted, and
the substitute ingredient information indicating the substitute ingredient and the quantity of the substitute ingredient is derived based on the at least one substitute ingredient that is selected and accepted.

12. An information provision system comprising:
a server that derives a substitute ingredient serving as a replacement for a standard ingredient, wherein
the server acquires information indicating the standard ingredient and a quantity of the standard ingredient and derives at least one substitute ingredient from reference ingredients and information indicating a quantity of the at least one substitute ingredient, based on the quantity of the standard ingredient, a content of a chemical component contained in the standard ingredient, and a content of a chemical component contained in each of the reference ingredients.

13. A recording medium storing a program that causes a computer to execute a process, the recording medium being non-volatile and causing the computer to execute the information provision method according to claim 1.

14. A cooking apparatus that uses the program according to claim 13.
